(19) United States
(12) Patent Application Publication
(10) Pub. No.: US 2003/0216470 A1
(43) Pub. Date: Nov. 20, 2003

(54) Method for treating ileus

(76) Inventor: Mitchell P. Fink, Pittsburgh, PA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/419,234

(22) Filed: Apr. 17, 2003

Related U.S. Application Data
(60) Provisional application No. 60/373,680, filed on Apr. 17, 2002.

(51) Int. Cl. A61K 31/22; A61K 31/19

(52) U.S. Cl. 514/547; 514/546; 514/557

(57) ABSTRACT

Disclosed is a method for treating ileus in a subject. The method comprises the step of administering to the subject an effective amount of an alpha-ketoalkanoic acid, a physiologically-acceptable salt of an alpha-ketoalkanoic acid, an ester of an alpha-ketoalkanoic acid, or an amide of an alpha-ketoalkanoic acid.

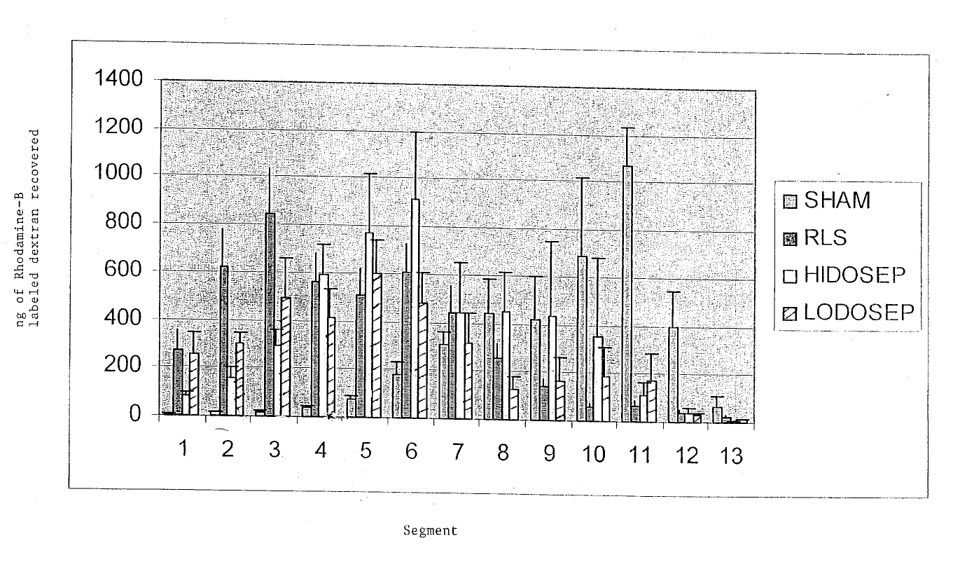

### RELATED APPLICATIONS

This application claims the benefit of U.S. Provisional Application No. 60/373,680, filed Apr. 17, 2002. The entire teachings of the above application are incorporated herein by reference.

### BACKGROUND OF THE INVENTION

Ileus is a partial or complete non-mechanical obstruction of the small and/or large intestine. Ileus occurs when peristalsis, the rhythmic contraction that moves material through the bowel, stops. Ileus can be caused, for example, by manipulation of the intestines during abdominal surgery, inflammation of the peritoneum, or administration of narcotics or chemotherapeutic agents.

While postoperative ileus usually resolves spontaneously within about 36 to 96 hours, until it resolves, supervised bed rest and bowel rest in a hospital is the current therapy. Patients with ileus take no food or medications by mouth. The patients are hydrated intravenously and gastric decompression is provided through the use of a nasogastric tube. The discomfort, inconvenience and economic costs of this current therapy are substantial.

Methods for preventing ileus are presently unavailable, and methods for treating ileus are currently inadequate. Thus, there is an urgent need for new methods of preventing and/or ameliorating the effects of ileus.

### SUMMARY OF THE INVENTION

It has been found that certain α-keto acids, α-keto esters and α-keto amides can be used to ameliorate the effects of ileus. For example, when Ringer's Ethyl Pyruvate Solution (REPS) was administered to laboratory rats prior to surgery, during which the bowels were manipulated, Ringer's Ethyl Pyruvate Solution decreased the occurrence of ileus, compared to control rats administered Ringer's Lactate Solution prior to bowel manipulation surgery (Example 2). Accordingly, disclosed herein is a method for treating subjects that have or are at risk for developing ileus. The method comprises administering to the subject an effective amount of an ester of an alpha-ketoalkanoic acid, an amide of an alpha-ketoalkanoic or an alpha-ketoalkanoic acid or salt thereof.

The method of the present invention has several advantages. The therapeutic or prophylactic treatment of ileus using the compounds described herein allows for recovery from surgery without the discomfort of having to be fed intravenously. In addition, by treating ileus as described herein, health care facilities can improve patient quality care because the time spent by the medical staff administering food and medications through non-oral routes can now be devoted to other patient care tasks. Medical supplies are also reduced by treating ileus using the method of the invention, and lengths of stay in the hospital after operation are reduced.

### BRIEF DESCRIPTION OF THE DRAWINGS

The FIGURE is a histogram of the mean dye concentration (±SEM) in each segment of the intestine after sham, administration of Ringer's Lactate Solution (RLS) followed by manipulation of the bowel, or administration of Low Dose Ringer's Ethyl Pyruvate Solution (LODOSEP) followed by bowel manipulation, or administration of High Dose Ringer's Ethyl Pyruvate Solution (HIDOSEP) followed by bowel manipulation in rats. Segment 1 is the small bowel segment that begins at the duodenal-jejunal junction (ligament of Treitz); segment 2 is the next segment moving along the bowel in the oral to anal direction. Segment 10 is the last segment of small bowel just proximal to the ileal cecal junction. Segments 11, 12 and 13 are the three colonic segments, corresponding roughly to the proximal colon (including the cecum), the midcolon, and the distal colon, respectively.

### DETAILED DESCRIPTION OF THE INVENTION

The present invention is directed to a method of treating ileus in a subject by administering an alpha-ketoalkanoic acid, a physiologically-acceptable salt of an alpha-ketoalkanoic acid, an ester of an alpha-ketoalkanoic acid, or an amide of an alpha-ketoalkanoic acid dissolved in a physiologically-acceptable vehicle. When the therapeutic agent is an ester of an alpha-ketoalkanoic acid, the pharmaceutical composition preferably comprises an enolization agent, and is dissolved in a physiologically-acceptable vehicle.

As used herein, by “ileus” is meant the arrest (stoppage or decreased activity) of intestinal peristalsis having causes other than interruption of blood flow to the intestines or by reperfusion in the intestines. Ileus can be detected, for example, by auscultation. Symptoms of ileus include, but are not limited to abdominal distention, vomiting, obstipation, cramps, hiccups, or gaseous distention of isolated segments of small and/or large bowel or colon, as detected by X-rays, computed tomography scans or ultrasound. Ileus can be caused, for example, by manipulation of the intestines during abdominal surgery, inflammation of the peritoneum, or administration of narcotics, for example, morphine sulfate, meperidine hydrochloride, codeine phosphate, or oxycodone hydrochloride, or chemotherapeutic agents such as vincristine, vinorelbine tartrate, doxorubicin hydrochloride or BCNU (carmustine).

The present invention features alpha-ketoalkanoic acids or physiologically-acceptable salts thereof, esters of alpha-ketoalkanoic acids, or amides of alpha-ketoalkanoic acids for use in treating ileus.

Suitable alpha-ketoalkanoic acids include C₃-C₈ straight chained or branched alpha-ketoalkanoic acids, for example, pyruvic acid. Physiologically acceptable salts, of α-ketoalkanoic acids include Na⁺, K⁺, Ca⁺⁺, Mg²⁺, NH₄⁺ and the like.

In one aspect, the therapeutic agent used in the method disclosed herein is an effective amount of an ester of an alpha-ketoalkanoic acid, for example, a C₃-C₈ straight-chained or branched alpha-ketoalkanoic acid. Examples include alpha-keto-butyrate, alpha-ketopentanoate, alpha-keto-3-methyl-butyrate, alpha-keto-4-methyl-pentanoate or alpha-keto-hexanoate. Pyruvate is preferred. A variety of groups are suitable for the ester position of the molecule, e.g., alkyl, aralkyl, alkoxyalkyl, carbalkoxyalkyl, glyceryl or dihydroxy acetone. Specific examples include ethyl, propyl, butyl, carbmethoxymethyl (—CH₂COOCH₃), carbethoxymethyl (—CH₂COOCH₂CH₃), acetoxymethyl (—CH₂OC(O)CH₃), carbmethoxyethyl (—CH₂CH₂COOCH₃), carbethoxyethyl (—CH₂CH₂COOCH₂CH₃), methoxymethyl (—CH₂OCH₃) and ethoxymethyl (—CH₂OCH₂CH₃). Ethyl esters are preferred. Thiolesters (e.g., wherein the thiol portion is cysteine or homocysteine) are also included.

In a preferred embodiment, the pharmaceutical composition used in the disclosed method comprises ethyl pyruvate, propyl pyruvate, carbmethoxymethyl pyruvate, acetoxymethyl pyruvate, carbethoxymethymethyl pyruvate, ethoxymethyl pyruvate, ethyl alpha-keto-butyrate, ethyl alpha-keto-pentanoate, ethyl alpha-keto-3-methyl-butyrate, ethyl alpha-keto-4-methyl-pentanoate, or ethyl alpha-keto-hexanoate. Ethyl pyruvate is more preferred.

In another aspect, the invention is a method of treating ileus occurring after abdominal surgery in a subject, comprising administering to the subject an effective amount of ethyl pyruvate, wherein the ethyl pyruvate is in a physiologically-acceptable carrier comprising a sufficient quantity of calcium or magnesium for inducing and/or stabilizing enolization of said ethyl pyruvate at physiological pH values, wherein the carrier is Ringer's Lactate-like solution in a pH range of about 4 to about 8, and preferably at a pH value of about 5 to about 7. Other carriers for the compounds of the present invention include isotonic salt solutions buffered with citrate, for example, approximately 100 mM to 200 mM citrate.

In yet another aspect, the therapeutic agent used in the method disclosed herein is an effective amount of an amide of an alpha-ketoalkanoic acid.

Suitable amides of alpha-ketoalkanoic acids for use in the method of the present inventions include compounds having the following structural formula: RCOCONR1R2. R is an alkyl group; R1 and R2 are independently —H, alkyl, aralkyl, alkoxyalkyl, carboxyalkyl or —CHR3COOH; and R3 is the side chain of a naturally occurring amino acid. Preferably, the amide of an alpha-ketoalkanoic acids is a pyruvamide.

Suitable alkyl groups include C₁-C₈ straight chained or branched alkyl group, preferably C₁-C₆ straight chained alkyl groups.

Suitable aryl groups include carbocyclic (e.g., phenyl and naphthyl) and heterocyclic (e.g., furanyl and thiophenyl) aromatic groups, preferably phenyl.

An alkoxy group is —OR4, wherein R4 is an alkyl group, as defined above. An alkoxyalkyl group is an alkyl group substituted with —OR4.

An aralkyl group is —XY, wherein X is an alkyl group and Y is an aryl group, both as defined above. Divalent cations are introduced into the pharmaceutical formulation as a salt, e.g., as calcium chloride or magnesium chloride.

A carboxyalkyl group is an alkyl group substituted with —COOH.

A carbalkoxyalkyl group is an alkyl group substituted with an ester.

The pharmaceutical compositions used in the method of the present invention preferably include an enolization agent when the therapeutic agent is an α-keto ester. The enolization agent and α-keto ester are in a physiologically acceptable carrier. An “enolization agent” is a chemical agent which induces and stabilizes the enol resonance form of an alpha-keto ester at or around physiological pH (e.g., between about 4.0 to about 8.0, more preferably between about 4.5 to about 6.5). Enolization agents include a cationic material, preferably a divalent cation such as calcium or magnesium or, for example, a cationic amino acid such ornithine or lysine. Divalent cations are introduced into the pharmaceutical formulation as a salt, e.g., as calcium chloride or magnesium chloride. Preferably, sufficient enolization agent is present in the pharmaceutical composition to stabilize the enol form. Stabilization of the enol form is indicated by an increase in solubility of the pyruvate ester in aqueous solution at or around physiological pH. For example, alpha keto esters such as pyruvate esters are generally only marginally soluble in aqueous solution at or around physiological pH, but the enol form of these esters can be dissolved to form solutions having a concentration between about 20 mM to about 200 mM. In the present application; the enol form is said to be “stabilized” in aqueous solution at pH between about 4 to about 8, for example, between about 4.5 to about 6.5, when sufficient enolization agent (typically between 1 mmole to 6 mmoles of enolization agent is present more typically between 2 mmoles to 3 mmoles) is present such that the concentration of alpha-ketoalkanoic acid ester dissolved in the solution is at least 20 mM.

The enolization agent significantly increases the solubility of the alpha-ketoalkanoic acid ester in aqueous solution. Therefore, pharmaceutical solutions containing the enolization agent can have higher concentrations of alpha-ketoalkanoic acids than pharmaceutical solutions without the enolization agent. The more concentrated pharmaceutical compositions are more convenient to use and provide an improved therapeutic benefit compared with the less concentrated solutions. Thus, the use of pharmaceutical compositions comprising an ester of an alpha-ketoalkanoic acid with an enolization agent provides for an improved method of treating ileus.

The terms “therapeutic” and “treatment” as used herein, refer to ameliorating symptoms associated with a disease or condition, including preventing, inhibiting or delaying the onset of the disease symptoms, and/or lessening the severity, duration or frequency of symptoms of the disease.

A “subject” is preferably a human patient, but can also be a companion animal (e.g., dog, cat and the like), a farm animal (e.g., horse, cow, sheep, and the like) or laboratory animal (e.g., rat, mouse, guinea pig, and the like). The method of the present invention is ideally suited to prophylactically treat subjects at risk for ileus, for example, a subject undergoing abdominal surgery, experiencing abdominal surgery, or being administered narcotics or chemotherapeutic agents.

Formulation of a therapeutic agent to be administered will vary according to the route of administration selected (e.g., solution, emulsion, capsule), and can be sterile if so desired. An appropriate composition comprising the agent to be administered can be prepared in a physiologically or pharmaceutically acceptable vehicle or carrier. A physiologically or pharmaceutically acceptable carrier for the composition used in the method of the present invention can be any carrier vehicle generally recognized as safe for administering a therapeutic agent to a mammal, e.g., a buffer solution for infusion, or bolus injection, a tablet for oral administration or in gel, micelle or liposome form for on-site delivery. A preferred buffer solution is water or isotonic or hypertonic saline buffered with bicarbonate, phosphate or citrate at 0.1M to 0.2M. Alternatively, the therapeutic agent is administered in a plasma extender, microcolloid or microcrystalline solution.

One preferred example of a formulation of a therapeutic agent for treating ileus is a Ringer's solution of isotonic saline supplemented with potassium ion (0 to about 4 milliequivalents/liter) and sodium (about 100 to about 156 milliequivalents/liter), for example, as described herein. Another preferred example is a citrated (0.1M to 0.2M) buffered solution with potassium ion (0 to about 4 milliequivalents/liter) and sodium ion (about 100 to 156 milliequivalents/liter). When ethyl pyruvate is the therapeutic agent, a preferred concentration range is from about 0.1 to about 10% by weight. In a particularly preferred aspect, the pharmaceutical composition comprises ethyl pyruvate (approximately 10 mg/ml) admixed with calcium chloride in a Ringer's solution at a pH in the range of about 4 to about 8 (REPS). In another aqueous formulation, the ethyl pyruvate is contained in a 0.2 M citrate buffer at pH of about 4 to about 5. Another aqueous formulation is as follows: 0.2 M citrate buffer, 10 mg/ml ethyl pyruvate (1%), 102 mM NaCl, 4 mM KCl, and 2.7 mM CaCl₂. Another formulation comprises 2% to 3% ethyl pyruvate, approximately 100 mM citrate buffer, 4 mM KCl, and 2.7 mM CaCl₂. The formulation administered for the treatment of ileus can be formed by admixing components of a two part formulation, one part containing, for example, ethyl pyruvate, (neat), and the second part consisting of the remaining components of a desired aqueous formulation, for example, those reagents described above.

The precise dose to be employed in the formulation of a therapeutic agent will depend on the route of administration, and the seriousness of the conditions, and should be decided according to the judgment of a practitioner and each patient's circumstances. Effective doses may be extrapolated from dose-response curves derived from in vitro or animal model test systems.

According to the method, an (i.e., one or more) alpha-ketoalkanoic acid or physiologically-acceptable salt thereof, an ester of an alpha-ketoalkanoic acid, or an amide of alpha-ketoalkanoic acid can be administered to a subject by an appropriate route, either alone or in combination with another drug. An effective amount of an alpha-ketoalkanoic acid or physiologically-acceptable salt thereof, an ester of an alpha-ketoalkanoic acid, or an amide of alpha-ketoalkanoic acid is administered. An effective amount is an amount sufficient to achieve the desired therapeutic or prophylactic effect, under the conditions of administration, such as an amount sufficient for treating (therapeutically or prophylactically) ileus.

The therapeutic compositions of the invention can be administered through a variety of routes, for example, oral, dietary, topical, intravenous, intramuscular, or by inhalation (e.g., intrabronchial, intranasal or oral inhalation, intranasal drops) routes of administration, depending on the agent and disease or condition to be treated, using routine methods in physiologically-acceptable inert carrier substances. Other suitable methods of administration can also include rechargeable or biodegradable devices, and slow release polymeric devices. For example, the therapeutic compositions can be administered in a sustained release formulation using a biodegradable biocompatible polymer, or by on-site delivery using micelles, gels, liposomes, or a buffer solution. Preferably, the pharmaceutical composition is administered as an infusate at a concentration of, e.g., 10 mM to 200 mM, preferably 20 mM to 90 mM of the active agent, at a rate of 1 mg/kg body weight/day to 200 mg/kg body weight/day, in a buffer solution as described herein. More preferably, the pharmaceutical composition is administered as an infusate at a concentration of about 28 mM of the active agent at a dose of 100 mg/kg body weight/day to 150 mg/kg body weight/day of alpha-ketoalkanoic acid, in a buffer solution. In bolus form, the active agent can be administered at a similar dosage, e.g., 1 mg/kg body weight/day to 200 mg/kg body weight/day of active agent, where the dosage is divided into aliquots and delivered 1 to 4 times daily (for a total dosage of 1 mg/kg body weight/day to 200 mg/kg body weight/day), with the concentration of the active agent adjusted accordingly. The enolization agent in the composition of the invention is at an appropriate concentration to induce enolization of the alpha-keto functionality of the amount of active ester agent in the administered composition. Optimal dosage and modes of administration can readily be determined by conventional protocols.

The method of the present invention can be used to treat ileus at the time of onset, and is also particularly suited for prophylactic treatment of ileus. “Prophylactic treatment” refers to treatment before onset of ileus to prevent, inhibit or reduce the occurrence of ileus. For example, a subject at risk for ileus, such as a subject undergoing abdominal surgery, or about to undergo abdominal surgery, or being (or about to be) administered narcotics or chemotherapeutic agents can be prophylactically treated according to the method of the present invention prior to the anticipated onset of ileus, (for example, prior to, during, an/or for up to about 48 hours after abdominal surgery, prior to or during administration of narcotics or chemotherapeutics, or at the onset of abdominal inflammation, but prior to the onset of ileus).

While this invention has been particularly shown and described with references to preferred embodiments thereof, it will be understood by those skilled in the art that various changes in form and details may be made therein without departing from the scope of the invention encompassed by the appended claims.

### EXEMPLIFICATION

The present invention will now be illustrated by the following Examples, which are not intended to be limiting in any way.

### Example 1

### An In Vivo Model for Testing Therapies for Treating Ileus Occurring After Abdominal Surgery

Inbred A X C 9935 Irish (ACI) male rats (200-250 g) were used to carry out the studies described below. The rats were anesthetized by methoxyflurane inhalation. The following operation was performed under sterile conditions, and the intestine was handled only with instruments. A midline vertical laparotomy incision was made on each rat. The entire small bowel was eventrated onto a saline-soaked gauze sponge. The small intestine was then manipulated from the proximal jejunum to the ileocecal junction by compressing the bowel progressively from the proximal to the distal end of the gut between two cotton applicators (e.g., “Q-tips”). This procedure was designed to be analogous to a surgeon “running” the bowel between his fingers to carefully examine its surfaces during an exploratory laparotomy procedure.

The bowel was returned to the peritoneal cavity, and the abdomen was closed in layers. The animals were returned to their cages and provided with access to food and water. A group of control rats undergoing a “sham” procedure received the anesthetic but were not subjected to either laparotomy or bowel manipulation.

Intestinal transit was measured at 23 hours (h) after the operation, and is described as follows. Animals were re-anesthetized as described above. Rhodamine B-labeled dextran-70 (mol wt=70,000 Da) was administered by gavage or gastric tube into the stomach (0.1 ml of a solution containing 25 mg/ml). The animals were then allowed to recover from anesthesia.

Two hours later, the animals were anesthetized again. The abdomen was opened and the entire small bowel was excised and divided into 10 equal segments in order to determine the length that the rhodamine B-labeled dextran-70 had progressed though the intestines (and therefore, the presence and/degree of severity of ileus). The large bowel was removed and divided into three segments, corresponding to cecum, proximal colon, and distal colon. The segments were mixed vigorously with 2 ml of saline solution. The cloudy suspension of intestinal contents and saline was centrifuged (1000×g for 15 minutes) to pellet the insoluble intestinal contents. The cleared solution from each segment was then assayed fluorometrically (excitation 530+/−20 nm, emission+/−50 nm). An agent that treats ileus is an agent that increases the length of bowel through which the rhodamine B-labeled dextran-70 progresses.

### Example 2

### The Effect of REPS on the Development of Ileus in Rats Following Bowel Manipulation

A study of the effects of REPS on the development of ileus in rats following bowel manipulation was carried out as follows. Inbred A X C 9935 Irish (ACI) male rats (200-250 g) rats were separated into four groups; one group (Sham control), which underwent anesthesia, but did not undergo laparotomy or bowel manipulation, another group (RLS control), underwent anesthesia, laparotomy, and bowel manipulation, and was administered Ringer's Lactate Solution, another group (LODOSEP) underwent anesthesia, laparotomy, and bowel manipulation, and was administered low dose REPS, as described herein, and a fourth group (HIDOSEP) underwent anesthesia, laparotomy, and bowel manipulation, and was administered high dose REPS, as described herein. There were 10 rats in each group.

The following solutions were made in preparation for determining the effect of administration of REPS on the development of ileus following bowel manipulation in rats: 10× Ringers Balanced Salt Solution (RBSS) (1.020 M NaCl, 40 mM KCl, and 27 mM CaCl₂.2H₂O); 10× sodium lactate (280 mM); and 10× NaCl (280 mM). These solutions were combined as described in Table 1 to prepare the following infusion solutions for administration to the rats: Ringer's Lactate Solution (RLS; control solution); Low Dose Ringer's Ethyl Pyruvate Solution (LODSEP) (containing 28 mM ethyl pyruvate) and High Dose Ringer's Ethyl Pyruvate Solution (HIDOSEP) (containing 84 mM ethyl pyruvate).

TABLE 1

| | Distilled | 10 × | 10 × sodium | | Ethyl |
|---|---|---|---|---|---|
| | H₂O | RBSS | lactate | 10 × NaCl | pyruvate |
| RLS | 40 ml | 5 ml | 5 ml | — | — |
| LODOSEP | 40 ml | 5 ml | — | 5 ml | 155 μL |
| HIDOSEP | 40 ml | 5 ml | — | 5 ml | 465 μL |

The rats were anesthetized as described in Example 1, and instrumented with a venous catheter. An infusion of normal saline at 1 ml/h was started to keep the catheter patent. Ten minutes of normal saline infusion were allowed for stabilization. Then, the rats were treated according to the schedules presented in Table 2.

TABLE 2

| Group | T = −5 min | T = 0 min | T = 90 min | T = +23 hours |
|---|---|---|---|---|
| Sham control | No intervention | No intervention | Stop i.v. infusion | Reanesthetize rat; |
| | | | and remove i.v. | harvest bowel |
| | | | catheter, | segments for |
| | | | | measurements of |
| | | | | rhodamine-dextran |
| | | | | transit. |
| RLS control | Inject 3 ml/kg | Perform | Stop i.v. infusion | Reanesthetize rat; |
| | bolus of RLS | laparotomy/bowel | and remove i.v. | harvest bowel |
| | and start infusion | manipulation, and | catheter. Return | segments for |
| | at 3 ml/kg per h. | then close | rat to cage. | measurements of |
| | | abdominal | | rhodamine-dextran |
| | | incision. | | transit. |
| LODOSEP | Inject 3 ml/kg | Perform | Stop i.v. infusion | Reanesthetize rat |
| | bolus of | laparotomy/bowel | and remove i.v. | harvest bowel |
| | LODOSEP and | manipulation, and | catheter. Return | segments for |
| | start infusion at | then close | rat to cage. | measurements of |
| | 3 ml/kg per h. | abdominal | | rhodamine-dextran |
| | | incision. | | transit. |
| HIDOSEP | Inject 3 ml/kg | Perform | Stop iv. infusion | Reanesthetize rat; |
| | bolus of | laparotomy/bowel | and remove i.v. | harvest bowel |
| | HIDOSEP and | manipulation, and | catheter. Return | segments for |
| | start infusion at | then close | rat to cage. | measurements of |
| | 3 ml/kg per h. | abdominal | | rhodamine-dextran |
| | | incision. | | transit. |

(N = for each group)

At T=+23 hours the rats are reanesthetized and Rhodamine B-labeled dextran-70 (mol wt ˜70,000 Da) was administered as described above. Then the bowel segments were harvested and the mean fluorescence intensity for each numbered segment in each treatment group was determined, as described in Example 1. The mean dye concentration in each segment of bowel is shown in the FIGURE. In the Sham control rats, there was little labeled dextran in segments 1-5, and the majority of the dextran was located in segments 10 and 11. In the rats that were administered RLS only, followed by bowel manipulation, labeled dextran was found predominantly in the first segments of the small bowel, and in particular in segments 2 and 3. Rats administered Low Dose REPS (LODOSEP) followed by bowel manipulation had labeled dextran in segments further toward the distal colon than those rats in the RLS group, with the highest amounts of labeled dextran found in segments 3-6. This data, indicates that Low Dose REPS is effective at treating ileus. In addition, rats administered High Dose REPS (HIDOSEP) followed by bowel manipulation had labeled dextran in segments even further toward the distal colon than those rats in the RLS group or the Low Dose REPS group, with segments 6 containing the highest amount of labeled dextran out of all the segments. These results again indicate that High Dose REPS is effective at treating ileus.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating ileus in a subject, said method comprising administering to said subject an effective amount of an ester of an alpha-ketoalkanoic acid, an amide of an alpha-ketoalkanoic or an alpha-ketoalkanoic acid or salt thereof.

2. A method of treating ileus occurring after abdominal surgery in a subject, said method comprising administering to said subject an effective amount of an alpha-ketoalkanoic acid or salt thereof.

3. The method of claim 2, wherein said alpha-ketoalkanoic acid is pyruvic acid or a physiologically-acceptable salt thereof.

4. The method of claim 2, wherein said alpha-ketoalkanoic acid is a C₃-C₈ straight chained or branched alpha-ketoalkanoic acid or a physiologically-acceptable salt thereof.

5. A method of treating ileus occurring after abdominal surgery in a subject, said method comprising administering to said subject an effective amount of an ester of an alpha-ketoalkanoic acid.

6. The method of claim 5, wherein said ester of an alpha-ketoalkanoic acid is a C₃-C₈ straight-chained or branched alpha-ketoalkanoic acid.

7. The method of claim 5, wherein said ester of an alpha-ketoalkanoic acid is an alkyl, aralkyl, alkoxyalkyl or carbalkoxyalkyl ester.

8. The method of claim 7, wherein said ester of an alpha-ketoalkanoic acid is an ethyl ester.

9. The method of claim 5, wherein said ester of an alpha-ketoalkanoic acid is an ester of pyruvic acid.

10. The method of claim 5, wherein said ester of an alpha-ketoalkanoic acid is ethyl pyruvate.

11. The method of claim 5, wherein said ester of an alpha-ketoalkanoic acid is contained in a physiologically-acceptable carrier.

12. The method of claim 5, wherein said ester of an alpha-ketoalkanoic acid is in a physiologically-acceptable carrier, said carrier further comprising a physiologically-acceptable enolization agent.

13. The method of claim 12, wherein said enolization agent is an inorganic, divalent cation.

14. The method of claim 13, wherein said divalent cation is calcium or magnesium.

15. The method of claim 13, wherein said ester of an alpha-ketoalkanoic acid portion is an ester of pyruvic acid.

16. The method of claim 6, wherein said alpha-ketoalkanoic acid ester is an ester of glycerol.

17. The method of claim 6, wherein said alpha-ketoalkanoic acid ester is an ester of dihydroxyacetone.

18. The method of claim 6, wherein said alpha-ketoalkanoic acid ester is a thiolester.

19. The method of claim 18, wherein said thiol portion of said thiolester is cysteine or homocysteine.

20. The method of claim 12, wherein said physiologically-acceptable carrier is Ringer's isotonic saline comprising potassium ion and/or sodium ion.

21. The method of claim 12, wherein said ester of an alpha-ketoalkanoic acid is selected from the group consisting of ethyl pyruvate, propyl pyruvate, carbmethoxymethyl pyruvate, acetoxymethyl pyruvate, carbethoxymethymethyl pyruvate, and ethoxymethyl pyruvate.

22. The method of claim 12, wherein said ester of an alpha-ketoalkanoic acid is selected from the group consisting of ethyl alpha-keto-butyrate, ethyl alpha-keto-pentanoate, ethyl alpha-keto-3-methyl-butyrate, ethyl alpha-keto-4-methyl-pentanoate, and ethyl alpha-keto-hexanoate.

23. The method of claim 21, wherein said ester of an alpha-ketoalkanoic acid is in a solution, said solution comprising a cation selected from the group consisting of calcium and magnesium.

24. The method of claim 22, wherein said ester of an alpha-ketoalkanoic acid is in a solution, said solution comprising a cation selected from the group consisting of calcium and magnesium.

25. A method of treating ileus occurring after abdominal surgery in a subject, said method comprising administering to said subject an effective amount of ethyl pyruvate, wherein said ethyl pyruvate is in a physiologically-acceptable carrier comprising a sufficient quantity of calcium or magnesium for inducing a stabilizing enolization of said ethyl pyruvate at physiological pH values.

26. A method of treating ileus occurring after abdominal surgery in a subject, said method comprising administering to said subject an effective amount of an amide of an alpha-ketoalkanoic acid.

27. The method of claim 26, wherein said amide of an alpha-ketoalkanoic acid portion is a pyruvamide.

28. The method of claim 26, wherein said amide of an alpha-ketoalkanoic acid portion is an amino acid amide.

29. A method of treating ileus resulting from inflammation of the abdomen in a subject, said method comprising administering to said subject a therapeutically effective amount of an amide of an ester of an alpha-ketoalkanoic acid, an amide of an alpha-ketoalkanoic acid, or an alpha-ketoalkanoic acid or a salt thereof.

30. A method of treating ileus occurring after administration of a narcotic or anti-cancer agent in a subject, said method comprising administering to said subject a therapeutically effective amount of an ester of an alpha-ketoalkanoic acid, an amide of an alpha-ketoalkanoic acid, or an alpha-ketoalkanoic acid or a salt thereof.
